(19) United States
(12) Patent Application Publication
(10) Pub. No.: US 2004/0033246 A1
(43) Pub. Date: Feb. 19, 2004

(54) Cosmetic for prevention of skin aging and agent for prevention of skin aging

(75) Inventors: Eiji Naru, Tokyo (JP); Ken Inomata, Tokyo (JP); Kazuhiko Kaji, Saitama-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KOSE CORPORATION, Tokyo (JP)

(21) Appl. No.: 10/178,511

(22) Filed: Jun. 25, 2002

(51) Int. Cl. A61K 35/78; A61K 35/72; A61K 31/739; A61K 31/4172; A61K 7/00; A61K 7/06

(52) U.S. Cl. 424/401; 424/195.16; 424/725; 514/54; 514/400

(57) ABSTRACT

There are disclosed a cosmetic for prevention of skin aging and an agent for prevention of skin aging containing the following components (A) and (B).

(A) L-histidine; and

(B) one or more active ingredient (s) selected from a group consisting of comfrey extract, yeast extract and ceramide.

In accordance with the present invention, there are provided a cosmetic for prevention of skin aging and an agent for prevention of skin aging achieving an excellent effect in the improvement of wrinkles and sagging of the skin caused by intrinsic aging, by exposure to active oxygen species such as singlet oxygen or to ultraviolet ray, etc.

### BACKGROUND OF THE INVENTION

1. Field of the Invention

The present invention relates to a cosmetic for prevention of skin aging and also to an agent for prevention of skin aging. More particularly, it relates to a cosmetic for prevention of skin aging and to an agent for prevention of skin aging in which lowering of cell metabolism function by aging and senility caused by chronic exposure to sunlight are prevented whereby an excellent effect of suppressing the aging or preventing the aging to the skin is achieved.

2. Description of the Related Art

Various active ingredients have been added already to cosmetics such as milky lotion, cream, lotion, facial pack, cleansing agent, essence and dispersed liquid to give a predetermined pharmaceutical effect. For example, in order to improve the senility such as wrinkles and sagging of the skin caused by aging and exposure to active oxygen or ultraviolet ray, a component such as vitamin A is added to cosmetics.

Actually however, there are many cases where the cosmetic to which such active ingredients are added has no sufficient anti-aging effect or does not achieve an expected pharmaceutical effect due to deterioration in the preparation and, therefore, there has been a demand for that.

In the meanwhile, it has been commonly known that cell senescence proceeds or is promoted by intrinsic aging or by generation of active oxygen, particularly, singlet oxygen. The present inventors have carried out an intensive investigation for the components which are to be used as anti-aging components for cosmetics and have previously found that L-histidine which is one of the essential amino acids and can be used as an ingredient of cosmetics (U.S. Pat. No. 5,916,542) has a suppressive action to lowering of metabolism of cells accompanied by intrinsic aging and also a high suppressive action for cell senescence where active oxygen such as singlet oxygen is eliminated (JP 2002-128651 A).

The present inventors have carried out further investigation for improving the suppressive action for cell senescence by L-histidine and, as a result, they have found that, when specific active ingredients are jointly used, better suppressive action for cell senescence is achieved and, in addition, there is no problem such as deterioration even when made into pharmaceutical preparations or the like whereupon the present invention has been achieved.

### SUMMARY OF THE INVENTION

Thus, the present invention provides a cosmetic for prevention of skin aging which is characterized in containing the following components (A) and (B),

(A) L-histidine: and

(B) one or more active ingredient(s) selected from a group consisting of comfrey extract, yeast extract and ceramide.

The present invention further provides an agent for prevention of skin aging containing the above-mentioned components (A) and (B).

The present invention still further provides a method for prevention of skin aging by application of a skin aging preventing agent containing the above-mentioned components (A) and (B) to the skin.

### DETAILED DESCRIPTION OF THE PREFERRED EMBODIMENTS

L-Histidine of the component (A) used in the present invention is a kind of basic α-amino acids and is a substance which has been known to have a suppressive action for cell senescence. In applying L-histidine of the component (A) to the cosmetic for prevention of skin aging according to the present invention, there is no particular limitation for its source, synthetic method, extracting method and purifying method but L-histidine which is prepared by commonly used various means may be used.

The amount of L-histidine which is the component (A) of the cosmetic for prevention of skin aging according to the present invention to the total amount of the cosmetic is preferably within a range of 0.001-5.0% by weight (hereinafter, just referred to as “%”) or, more preferably, within a range of 0.01-1.0% by weight. When the amount of the component (A) is within the said range, it is possible to give a cosmetic for prevention of skin aging having an excellent anti-aging effect when used together with the pharmaceutical components of the component (B) which will be mentioned later.

The component (B) which is used together with the component (A) in the cosmetic for prevention of skin aging according to the present invention is a pharmaceutical component selected from a group consisting of comfrey extract, yeast extract and ceramide.

Firstly, the comfrey extract used as one of the component (B) is an extract of comfrey which is a plant belonging to family Borraginaceae, genus Symphytum (botanical name: Symphytum officinale L.) with a solvent, and comfrey has been known as a perennial plant native to Europe being cultivated as foods and medicines.

In preparing the comfrey extract which is used in the cosmetic for prevention of skin aging according to the present invention, there is no particular limitation for the extracting part, solvent for extracting, extracting method, etc. for comfrey and, for example, any of whole plant, leaves, flowers, roots and stems of comfrey may be extracted. Thus, they are appropriately dried, finely cut, compressed or fermented and may be subjected to means such as extracting with an extracting solvent at any temperature, namely, at low temperature or at from room temperature to under heating.

Not applying only to water, examples of the extracting solvent for the extraction of comfrey are lower monohydric alcohol such as methyl alcohol, ethyl alcohol, 1-propanol, 2-propanol, 1-butanol and 2-butanol; liquid polyhydric alcohol such as glycerol, propylene glycol and 1,3-butylene glycol; lower alkyl ester such as ethyl acetate; hydrocarbon such as benzene, hexane and pentane; ketone such as acetone and methyl ethyl ketone; ether such as diethyl ether, tetrahydrofuran and dipropyl ether; acetonitrile; etc. and one of them may used solely or two or more thereof may be used jointly.

The extract obtained by the extraction may be appropriately filtered or subjected to a treatment such as adsorption, decolorization and purification using ion-exchange resin to make into a form of solution, paste, gel or powder. Further and if necessary, purifying treatment such as deodorization and decolorization may be carried out within such an extent that the advantage of the present invention is not deteriorated. An example thereof is that leaves of comfrey are extracted with 1,3-butylene glycol followed by filtering.

Amount of the comfrey extract in the cosmetic for prevention of skin aging according to the present invention is preferably within a range of 0. 001-5.0% or, more preferably, within a range of 0.01-2.0% to the total amount of the cosmetic. When the amount of the comfrey extract is within such a range, the extract does not affect the stability of the cosmetic and the component (A) in the cosmetic with a lapse of time when jointly used with L-histidine which is the component (A) resulting a cosmetic for prevention of skin aging having better suppressing effect for aging. When the comfrey extract is used in a form of an extract per se, there is no limitation for the concentration of the extract so far as the dried solid amount of the said extract is within the above-mentioned range.

With regard to the yeast extract which one of the component (B) of the present invention, an extract where yeast is extracted with a solvent may be exemplified. Examples of the yeast which can be used in the preparation of the extract are Candida utilis and Sacchromyces cerevisiae.

There is no particular limitation for the method for the preparation of the yeast extract used in the present invention and, for example, yeast is collected, made in a slurry and washed with water, cells are collected, disintegrated and extracted with an extracting solvent and the extract is subjected to purifying operations such as centrifugal separation, precise filtration and concentration in vacuo whereupon a yeast extract can be obtained. If necessary, it is further purified by means of adsorption, decolorization, etc. using ion-exchange resin or the like within such an extent that the advantage of the present invention is not deteriorated to give an extract in a form of solution, paste, gel or powder.

In addition to water, examples of the extracting solvent for preparing the yeast extract are lower monohydric alcohol such as methyl alcohol, ethyl alcohol, propyl alcohol and butyl alcohol; liquid polyhydric alcohol such as propylene glycol, butylene glycol and glycerol; ether such as diethyl ether and tetrahydrofuran; ketone such as acetone; etc. and one of them may used solely or two or more thereof may be used jointly.

With regard to the yeast extract, in addition to the above-mentioned extract where yeast is extracted with a solvent, there may be used an extract where enzyme is subjected to lysis by means of autolysis, acidic hydrolysis or enzymatic decomposition and the resulting lysis liquid is filtered or an extract where the said lysis liquid is dried and the dried substance is extracted with a solvent.

Amount of the yeast extract in the cosmetic for prevention of skin aging according to the present invention is preferably within a range of 0.001-5.0% or, more preferably, within a range of 0.01-2.0% to the total amount of the cosmetic. When the amount of the yeast extract is within such a range, the extract does not affect the stability of the cosmetic and the component (A) in the cosmetic with a lapse of time when jointly used with L-histidine which is the component (A) resulting a cosmetic for prevention of skin aging having better suppressing effect for aging. When the yeast extract is used in a form of an extract per se, there is no limitation for the concentration of the extract so far as the dried solid amount of the said extract is within the above-mentioned range.

With regard to the ceramide which is used as one of the component (B) according to the present invention, there may be used natural ceramide, synthetic ceramide, glyco-ceramide, a derivative thereof, etc. It is also possible to use a substance having a structure similar to ceramide.

Among the above, examples of the natural ceramide are ceramide extracted from cattle and horse and ceramide produced by microorganisms and more specific examples are types 1-6 ceramides such as ceramide I, ceramide II, ceramide III and ceramide IIIb, etc.

With regard to the synthetic ceramide, there may be exemplified a ceramide having a structure which is the same as that of naturally-occurring ceramide. With regard to the glyco-ceramide, there may be exemplified ceramides derived from wheat, rice bran and microorganism having glucose, mannose and galactose.

With regard to the above-mentioned ceramides used in the present invention, one of them may be used solely or two or more thereof may be used jointly.

Amount of the ceramide in the cosmetic for prevention of skin aging according to the present invention is preferably within a range of 0.001-5.0% or, more preferably, within a range of 0.01-2.0% to the total amount of the cosmetic. When the amount of the ceramide is within such a range, the extract does not affect the stability of the cosmetic and the component (A) in the cosmetic with a lapse of time when Jointly used with L-histidine which is the component (A) resulting a cosmetic for prevention of skin aging having better suppressing effect for aging.

The cosmetic for prevention of skin aging according to the present invention is prepared in such a manner that L-histidine which is the component (A) is compounded with at least one of the three types of the component (B)—comfrey extract, yeast extract and ceramide—and, as a result, it Is possible to afford the cosmetic for prevention of skin aging having better aging suppressing effect as compared with the case where L-histidine is solely used.

With regard to the most preferred embodiment of the cosmetic for prevention of skin aging according to the present invention, there may be exemplified the cosmetic for prevention of skin aging in which L-histidine which is the component (A) is compounded with all of the three components of comfrey extract, yeast extract and ceramide whereby the aging suppressing effect can be achieved to the highest extent.

The amount of the component (B) as a whole in the cosmetic for prevention of skin aging according to the present invention is preferably within a range of 0.001-10% or, more preferably. within a range of 0.03-4% to the total amount of the cosmetic regardless of the type of the pharmaceutical components constituting the component (B).

In the cosmetic for prevention of skin aging according to the present invention, the components (A) and (B) which are essential are combined and made into various common forms known in cosmetics according to conventional methods.

In preparing the cosmetic for prevention of skin aging, it is possible depending upon the form thereof that, in addition to the above-mentioned essential components, components which have been commonly used for cosmetics and pharmaceuticals which are applied to skin may be appropriately added and their examples are water (such as pure water, hot spring water and deep sea water, etc.), oily agent, surface-active agent, gelling agent, alcohol, water-soluble polymer, film-forming agent, resin, inclusion compound, pH adjusting agent, extract derived from plants, animals and microorganisms other than the components (A) and (B) of the present invention, enzyme, hormone and vitamin Specific examples of such optional components will be as follows.

Examples of the oily agent are squalane, liquid paraffin. liquid isoparaffin, cetostearyl alcohol, cholesterol, phytosterol, POE cholesterol ether, cetyl 2-ethylhexanoate, isostearic acid, cholesteryl 12-hydrodxystearate, dipentaerythritol fatty acid ester, dimethylpolysiloxane, decamethylcyclopentasiloxane and methylphenylpolysiloxane and one of them may be used solely or two or more thereof may be used jointly.

Examples of the surface-active agent are N-acylamino acid type active agent, sorbitan fatty acid ester, polyoxyethylene sorbitan fatty acid ester, polyoxyethylene sorbitol fatty acid ester, sucrose fatty acid ester, polyoxyethylene hydrogenated castor oil, polyoxyalkylenemodified organopolysiloxane, polyoxyalkylene-alkyl co-modified organopolysiloxane and alkanolamide and one of them may be used solely or two or more thereof may be used jointly.

Examples of the alcohol are lower alcohol such as ethyl alcohol and isopropanol and polyhydric alcohol such as glycerol, diglycerol, ethylene glycol, diethylene glycol, triethylene glycol, propylene glycol, dipropylene glycol, 1,3-butylene glycol and polyethylene glycol and one of them may be used solely or two or more thereof may be used jointly.

Examples of the water-soluble polymer are carob gum, pectin, algae colloid, locust bean gum, xanthan gum, methylcellulose, carboxyvinyl polymer, alkyl-modified carboxyvinyl polymer, polyoxyethylene polyoxypropylene copolymer and sodium polyacrylate. A film forming agent such as polyvinyl alcohol and polyvinylpyrrolidone is included therein as well. With regard to such water-soluble polymer, one of them may be used solely or two or more thereof may be used jointly.

Examples of the pH adjusting agent are lactic acid, sodium lactate, potassium carbonate, sodium hydrogen carbonate, ammonium hydrogen carbonate, sodium citrate, sodium malate, sodium oxalate and sodium hydroxide and one of them may be used solely or two or more thereof may be used jointly.

There is no particular limitation for the cosmetic for prevention of skin aging according to the present invention and, for example, it can be made into various cosmetics such as milky lotion, cream, lotion, facial pack, cleansing agent, makeup cosmetic and dispersed liquid. Among them, in view of the fact that the advantage of the present invention can be well achieved, a cosmetic of an emulsified type such as milky lotion and cream is preferred.

It is also possible that the above-mentioned component (A) and component (B) are combined to use as an agent for prevention of skin aging. Various embodiments of preparing method for such an agent for prevention of skin aging and preparing method and amount of each component may be carried out in accordance with those of the above-mentioned cosmetic for prevention of skin aging. The agent for prevention of skin aging can be made into various forms which are applied to various cosmetics.

### EXAMPLES

The present invention will now be illustrated in more detail by way of the following Examples although the present invention is not limited thereto at all.

### Example 1

Creams:

Creams of the present invention products 1-4 and comparative products 1-5 were prepared according to the formulations shown In Table 1 and the following manufacturing method. As a referential product, there was prepared a cream (referential product 1) where only L-histidine which Is the component (A) was compounded.

(Formulations)

TABLE 1

| | | Products of Invention | | | | Comparative Products | | | | | R [symbol] |
|---|---|---|---|---|---|---|---|---|---|---|---|
| Components (% by weight) | | 1 | 2 | 3 | 4 | 1 | 2 | 3 | 4 | 5 | 1 [symbol] |
| (1) | Beeswax | 6.0 | 6.0 | 6.0 | 6.0 | 6.0 | 6.0 | 6.0 | 6.0 | 6.0 | 6.0 |
| (2) | Cetanol | 5.0 | 5.0 | 5.0 | 5.0 | 5.0 | 5.0 | 5.0 | 5.0 | 5.0 | 5.0 |
| (3) | Reduced lanolin | 5.0 | 5.0 | 5.0 | 5.0 | 5.0 | 5.0 | 5.0 | 5.0 | 5.0 | 5.0 |
| (4) | Squalane | 30.0 | 30.0 | 30.0 | 30.0 | 30.0 | 30.0 | 30.0 | 30.0 | 30.0 | 30.0 |
| (5) | Lipophilic glyceryl monostearate | 4.0 | 4.0 | 4.0 | 4.0 | 4.0 | 4.0 | 4.0 | 4.0 | 4.0 | 4.0 |
| (6) | POE sorbitan monolaurate (20 E.O.) | 2.0 | 2.0 | 2.0 | 2.0 | 2.0 | 2.0 | 2.0 | 2.0 | 2.0 | 2.0 |
| (7) | L-Histidine *¹ | 0.25 | 0.25 | 0.25 | 0.25 | — | — | — | — | — | 0. [symbol] |
| (8) | Comfrey extract *² | 0.5 | — | — | 0.2 | — | 1.0 | — | — | — | — |
| (9) | Ceramide *³ | — | 0.5 | — | 0.2 | — | — | 1.0 | — | — | — |
| (10) | Yeast extract *⁴ | — | — | 0.5 | 0.2 | — | — | — | 1.0 | — | — |
| (11) | Dehydroretinal *⁵ | — | — | — | — | — | — | — | — | 1.0 | — |
| (12) | Preservative | q.s. | q.s. | q.s. | q.s. | q.s. | q.s. | q.s. | q.s. | q.s. | q.s. |
| (13) | Perfume | q.s. | q.s. | q.s. | q.s. | q.s. | q.s. | q.s. | q.s. | q.s. | q.s. |
| (14) | Water | bal | bal | bal | bal | bal | bal | bal | bal | bal | bal |

R.P.: referential product
q.s.: quantum sufficit
bal: balance
*¹ manufactured by Wako Pure Chemical
*² manufactured by Ichimaru Pharcos
*³ manufactured by Cosmoferm
*⁴ manufactured by BIO-DELL
*⁵ manufactured by Sigma

(Manufacturing Method)

A: The components (1)-(6), (9), (11) and (12) were mixed and heated to keep at 70° C.

B: A part of the component (14) was heated to keep at 70° C.

C: “B” was added to “A” and mixed with the components (7), (8) and (10) dissolved in the residue of the component (14). After that, the component (13) was added followed by cooling to give a cream.

The creams prepared by the above-mentioned formulations and manufacturing method were tested in terms of the improving effect for wrinkles of aged hairless mice according to the following test method. The result is shown in Table 2.

(Test Method)

A group comprising ten hairless mice (HOS/HR-1; six weeks age; female) was used as a test group. First, those hairless mice were preliminarily bred for one week for acclimation to the environment. After the preliminary breeding, the cream to be tested was applied to the back of the mice at the rate of 0.1 g/cm² twice daily (morning and night) for 20 weeks and, once daily, the mice were irradiated with UV (UV-A and B) with an intensity of 0.1 J/cm² (incidentally, before the irradiation, skin of the back was wiped to remove the remaining cream). The operation was continued for 20 weeks and the resulting effect for improving the wrinkles by the cream to be tested was evaluated according to the following criteria. Mean value of sum of the score for each mouse was calculated and evaluated. The result is shown in Table 2.

(Criteria for Evaluation)

| Evaluation | Score | Remark |
|---|---|---|
| very effective | 3 | nearly no wrinkle was noted on the skin |
| effective | 2 | wrinkles on the skin was not so conspicuous |
| a bit effective | 1 | wrinkles on the skin was a bit conspicuous |
| ineffective | 0 | wrinkles on the skin was conspicuous |

(Result)

TABLE 2

| | Products of the | | | | | | | | | |
|---|---|---|---|---|---|---|---|---|---|---|
| | Invention | | | | Comparative Products | | | | | R.P. |
| | 1 | 2 | 3 | 4 | 1 | 2 | 3 | 4 | 5 | 1 |
| Mean Value of the | 2.1 | 2.3 | 2.2 | 2.9 | 0.5 | 1.2 | 1.4 | 1.7 | 1.3 | 1.9 |
| Scores | | | | | | | | | | |

R.P.: referential product

As will be apparent from the result of Table 2. it was confirmed that the creams of the present invention where L-histidine which is the component (A) was jointly used with comfrey extract, yeast extract or ceramide which are the component (B) showed a high suppressive effect to generation of wrinkles as senility on the back of mice. Among them, the highest suppressive effect for wrinkles on the back of mice was achieved by the product 4 of the present invention where the three components of comfrey extract, yeast extract and ceramide were combined with L-histidine.

### Example 2

Creams:

Creams of the present invention products 5-9 and comparative products 6-9 were prepared according to the formulations shown in Table 3 and the following manufacturing method. As a referential product, there was prepared a cream (referential product 2) where only L-histidine which is the component (A) was compounded.

(Formulations)

TABLE 3

| | | Products of the Invention | | | | | Comparative Products | | | | R.P. |
|---|---|---|---|---|---|---|---|---|---|---|---|
| Components (% by weight) | | 5 | 6 | 7 | 8 | 9 | 6 | 7 | 8 | 9 | 2 |
| (1) | Beeswax | 4.0 | 4.0 | 4.0 | 4.0 | 4.0 | 4.0 | 4.0 | 4.0 | 4.0 | 4.0 |
| (2) | Cetanol | 5.0 | 5.0 | 5.0 | 5.0 | 5.0 | 5.0 | 5.0 | 5.0 | 5.0 | 5.0 |
| (3) | Reduced lanolin | 5.0 | 5.0 | 5.0 | 5.0 | 5.0 | 5.0 | 5.0 | 5.0 | 5.0 | 5.0 |
| (4) | Squalane | 20.0 | 20.0 | 20.0 | 20.0 | 20.0 | 20.0 | 20.0 | 20.0 | 20.0 | 20.0 |
| (5) | Lipophilic glyceryl monostearate | 4.0 | 4.0 | 4.0 | 4.0 | 4.0 | 4.0 | 4.0 | 4.0 | 4.0 | 4.0 |
| (6) | POE sorbitan monolaurate (20 E.O.) | 2.0 | 2.0 | 2.0 | 2.0 | 2.0 | 2.0 | 2.0 | 2.0 | 2.0 | 2.0 |
| (7) | L-Histidine *¹ | 0.10 | 0.10 | 0.10 | 0.10 | 0.10 | — | — | — | — | 0.5 |
| (8) | Comfrey extract *² | 0.05 | — | — | 0.05 | — | — | 1.00 | — | — | — |
| (9) | Yeast extract *⁴ | — | 0.05 | — | 0.10 | 0.10 | — | — | 1.00 | — | — |
| (10) | Ceramide *³ | — | — | 0.05 | 0.05 | 0.05 | — | — | — | 0.5 | — |
| (11) | Preservative | q.s. | q.s. | q.s. | q.s. | q.s. | q.s. | q.s. | q.s. | q.s. | q.s. |
| (12) | Perfume | q.s. | q.s. | q.s. | q.s. | q.s. | q.s. | q.s. | q.s. | q.s. | q.s. |
| (13) | Water | bal | bal | bal | bal | bal | bal | bal | bal | bal | bal |

R.P.: referential product
q.s.: quantum sufficit
bal: balance
*¹ manufactured by Wako Pure Chemical
*² manufactured by Ichimaru Pharcos
*³ manufactured by Cosmoferm
*⁴ manufactured by BIO-DELL

(Manufacturing Method)

A: The components (1)-(6), (10) and (11) were mixed and heated to keep at 70° C.

B: A part of the component (13) was heated to keep at 70° C.

C: “B” was added to “A” and mixed with the components (7)-(9) dissolved in the remainder of the component (13). After that, the component (12) was added followed by cooling to give a cream.

The creams prepared by the above-mentioned formulations and manufacturing method were tested in terms of the improving effect for wrinkles according to the following test method. The result is shown in Table 4.

(Test Method)

Ten ladies of 40-59 years age used the cream to their faces twice dally (morning and night) for 20 weeks. Effect of improving the wrinkles by application of the cream was evaluated according to the following criteria.

(Criteria for Evaluation)

| Evaluation | Remark |
|---|---|
| Effective | Wrinkles on the skin was not conspicuous |
| A bit effective | Wrinkles on the skin was not so conspicuous |
| Ineffective | No change as compared with before use |

(Result)

TABLE 4

| | Products of the | | | | | Comparative | | | | |
|---|---|---|---|---|---|---|---|---|---|---|
| | Invention | | | | | Products | | | | R.P. |
| | 5 | 6 | 7 | 8 | 9 | 6 | 7 | 8 | 9 | 2 |
| Effective | 7 | 7 | 7 | 10 | 8 | 0 | 2 | 3 | 4 | 6 |
| A bit effective | 3 | 3 | 3 | 0 | 2 | 2 | 2 | 2 | 2 | 4 |
| Ineffective | 0 | 0 | 0 | 0 | 0 | 8 | 6 | 5 | 4 | 0 |

As will be apparent from the result of Table 4, it was confirmed that the creams of the present invention where L-histidine which is the component (A) was jointly used with comfrey extract, yeast extract or ceramide which are the component (B) showed a high suppressive effect to generation of wrinkles of the skin whereby a firm and beautiful skin was able to be resulted. Among them, the suppressive effect for wrinkles on the skin by the cream of the present invention product 8 where the three components of comfrey extract, yeast extract and ceramide were combined with L-histidine showed the particularly good improving effect to the wrinkles on the skin whereby more firm and beautiful skin was able to be resulted.

### Example 3

Lotion:

A Lotion was prepared according to the following formulation and manufacturing method.

(Formulation)

| Components | % |
|---|---|
| (1) Glycerol | 5.0 |
| (2) 1,3-Butylene glycol | 6.5 |
| (3) Polyoxyethylene (20 E. O.) sorbitan | 1.2 |
| monolaurate | |
| (4) Ethyl alcohol | 8.0 |
| (5) L-Histidine*¹ | 0.3 |
| (6) Citric acid | 0.1 |
| (7) Sodium citrate | 0.1 |
| (8) Comfrey extract*² | 0.2 |
| (9) Preservative | q.s. |
| (10) Perfume | q.s. |
| (11) Water | balance |

*¹manufactured by Wako Pure Chemical
*²manufactured by Ichimaru Pharcos

(Manufacturing Method)

A. The components (3), (4), (9) and (10) were mixed and dissolved.

B. The components (1), (2), (5)-(8) and (11) were mixed and dissolved.

C. “A” and “B” were mixed homogeneously to manufacture a lotion.

The lotion obtained by Example 3 was excellent in terms of stability with a lapse of time and, when it was applied to the skin, wrinkles and sagging of the skin by aging were improved to make skin firm and beautiful.

### Example 4

Lotion:

A Lotion was prepared according to the following formulation and manufacturing method.

(Formulation)

| Components | % |
|---|---|
| (1) Glycerol | 5.0 |
| (2) 1,3-Butylene glycol | 6.5 |
| (3) Polyoxyethylene (20 E. O.) sorbitan | 1.2 |
| monolaurate | |
| (4) Ethyl alcohol | 8.0 |
| (5) Ceramide *³ | 0.05 |
| (6) L-Histidine *¹ | 0.3 |
| (7) Dipotassium glycyrrhizinate | 0.1 |
| (8) Yeast extract *⁴ | 0.5 |
| (9) Citric acid | 0.1 |
| (10) Sodium citrate | 0.1 |
| (11) Preservative | q.s. |
| (12) Perfume | q.s. |
| (13) Water | balance |

*¹manufactured by Wako Pure Chemical
*³manufactured by Cosmoferm
*⁴manufactured by BIO-DELL

(Manufacturing Method)

A. The components (3), (4), (5), (11) and (12) were mixed and dissolved.

B. The components (1), (2), (6)-(10) and (13) were mixed and dissolved.

C. “A” and “B” were mixed homogeneously to manufacture a lotion.

The lotion obtained by Example 4 was excellent in terms of stability with a lapse of time and, when it was applied to the skin, wrinkles and sagging of the skin by aging were improved to make skin firm and beautiful.

### Example 5

Milky Lotion:

A milky lotion was prepared according to the following formulation and manufacturing method.

(Formulation)

| Components | % |
|---|---|
| (1) Polyoxyethylene (10 E. O.) sorbitan | 1.0 |
| monostearate | |
| (2) Polyoxyethylene (60 E. O.) sorbitol | 0.5 |
| tetraoleate | |
| (3) Glyceryl monostearate | 1.0 |
| (4) Stearic acid | 0.5 |
| (5) Behenyl alcohol | 0.5 |
| (6) Squalane | 8.0 |
| (7) 2-Ethylhexyl p-methoxycinnamate | 3.0 |
| (8) Lycopene | 1.0 |
| (9) L-Histidine *¹ | 0.15 |
| (10) Comfrey extract *² | 0.1 |
| (11) Yeast extract *⁴ | 0.1 |
| (12) Ceramide *³ | 0.1 |
| (13) Preservative | 0.1 |
| (14) Carboxyvinyl polymer (CARBOPOL ® 940 *⁶) | 0.1 |
| (15) Sodium hydroxide | 0.05 |
| (16) Ethyl alcohol | 5.0 |
| (17) Water | balance |
| (18) Perfume | q.s. |
| (19) Sucrose fatty acid ester(DK-ester S-160 *⁷) | 0.5 |
| (20) α-Tocopherol | 0.1 |

*¹manufactured by Wako Pure Chemical
*²manufactured by Ichimaru Pharcos
*³manufactured by Cosmoferm
*⁴manufactured by BIO-DELL
*⁶manufactured by B. F. Goodrich
*⁷manufactured by Dai-ich Kogyo Seiyaku

(Manufacturing Method)

A. The components (14)-(16) and a part of (17) were mixed and heated to keep at 70° C.

B. The components (1)-(8), (12), (13), (19) and (20) were mixed and heated to keep at 70° C.

C. “A” was added to “B” to mix and homogeneously emulsify.

D. To cooled “C” were added the components (9)-(11) dissolved in the residual component (17), then (18) was added thereto and the mixture was homogeneously mixed to prepare a milky lotion.

The milky lotion obtained by Example 5 was excellent in terms of stability with a lapse of time and, when it was applied to the skin, wrinkles and sagging of the skin by aging were improved to make skin firm and beautiful.

### Example 6

Milky Lotion:

A milky lotion was prepared according to the following formulation and manufacturing method.

(Formulation)

| Components | % |
|---|---|
| (1) Polyoxyethylene (10 E. O.) sorbitan | 1.0 |
| monostearate | |
| (2) Polyoxyethylene (60 E. O.) sorbitol | 0.5 |
| tetraoleate | |
| (3) Glyceryl monostearate | 1.0 |
| (4) Stearic acid | 0.5 |
| (5) Behenyl alcohol | 0.5 |
| (6) Squalane | 8.0 |
| (7) 2-Ethylhexyl p-methoxycinnamate | 3.0 |
| (8) Ceramide *³ | 0.5 |
| (9) L-Histidine *¹ | 1.0 |
| (10) Comfrey extract *² | 0.2 |
| (11) Yeast extract *⁴ | 0.2 |
| (12) Preservative | 0.1 |
| (13) Acrylates/C-30 Alkyl Acrylate crosspolymer | 0.1 |
| (CARBOPOL ® 1342 *⁸) | |
| (14) Sodium hydroxide | 0.05 |
| (15) Ethyl alcohol | 5.0 |
| (16) Water | balance |
| (17) Perfume | q.s. |
| (18) Sucrose fatty acid ester(DK-ester S-160 *⁷) | 0.5 |

*¹manufactured by Wako Pure Chemical
*²manufactured by Ichimaru Pharcos
*³manufactured by Cosmoferm
*⁴manufactured by BIO-DELL
*⁷manufactured by Dai-ich Kogyo Seiyaku
*⁸manufactured by B.F. Goodrich

(Manufacturing Method)

A. The components (13)-(15) and a part of (16) were mixed and heated to keep at 70° C.

B. The components (1)-(8),(12) and (18) were mixed and heated to keep at 70° C.

C. “A” was added to “B” to mix and homogeneously emulsify.

D. To cooled “C” were added the components (9)-(11) and (17) dissolved in the residual component (16) followed by homogeneously mixing to prepare a milky lotion.

The milky lotion obtained by Example 6 was excellent in terms of stability with a lapse of time and, when it was applied to the skin, wrinkles and sagging of the skin by aging were improved to make skin firm and beautiful.

### Example 7

Facial Pack:

Facial pack was manufactured according to the following formulation and manufacturing method.

(Formulation)

| Components | % |
|---|---|
| (1) Polyvinyl alcohol | 20.0 |
| (2) Ethyl alcohol | 20.0 |
| (3) Glycerol | 5.0 |
| (4) Kaolin | 6.0 |
| (5) L-Histidine *¹ | 1.0 |
| (6) Sage extract | 2.0 |
| (7) Comfrey extract *2 | 3.0 |
| (8) Phytic acid | 0.05 |
| (9) Preservative | 0.2 |
| (10) Perfume | 0.1 |
| (11) Water | balance |

*¹manufactured by Wako Pure Chemical
*²manufactured by Ichimaru Pharcos

(Manufacturing Method)

A. The components (1)-(4) and (9) were mixed, a part of the component (11) heated at 70° C. was added thereto and the mixture was stirred.

B. “A” was cooled, the components (5)-(8) dissolved in the residual (11) were added thereto and the component (10) was homogeneously dispersed therein to give a facial pack.

The facial pack obtained by Example 7 was excellent in terms of stability with a lapse of time and, when it was applied to and taken off from the skin, wrinkles and sagging of the skin by aging were improved to make skin firm and beautiful.

### Example 8

Liquid Foundation:

A liquid foundation was manufactured according to the following formulation and manufacturing method.

(Formulation)

| Components | % |
|---|---|
| (1) Lanolin | 7.0 |
| (2) Liquid paraffin | 5.0 |
| (3) Stearic acid | 2.0 |
| (4) Cetanol | 1.0 |
| (5) L-Histidine *¹ | 0.5 |
| (6) 2-Ethylhexyl p-methoxycinnamate | 3.0 |
| (7) Glycerol | 5.0 |
| (8) Triethanolamine | 1.0 |
| (9) Carboxymethyl cellulose | 0.7 |
| (10) Water | balance |
| (11) Titanium dioxide | 8.0 |
| (12) Fine particles of titanium dioxide | 2.0 |
| (13) Zinc oxide | 5.0 |
| (14) Mica | 15.0 |
| (15) Talc | 6.0 |
| (16) Coloring pigments | 6.0 |
| (17) Astaxanthin | 0.01 |
| (18) Stearyl glycyrrhetinate | 0.1 |
| (19) Comfrey extract *² | 0.1 |
| (20) Citric acid | 0.1 |
| (21) Perfume | q.s. |

*¹ manufactured by Wako Pure Chemical
*² manufactured by Ichimaru Pharcos

(Manufacturing Method)

A. The components (1)-(4), (6), (17) and (18) were heated to dissolve.

B. The components (11)-(16) were added to “A” followed by mixing homogeneously to keep at 70° C.

C. The components (7)-(9) and a part of the component (10) were homogeneously dissolved to keep at 70° C.

D. “C” was added to “B” followed by homogeneously emulsifying.

E. To the cooled “D” were added (5), (19), (20) and (21) dissolved In the residual (10) to give a liquid foundation.

The liquid foundation obtained by Example 8 was excellent in terms of stability with a lapse of time and, when it was applied to the skin, wrinkles and sagging of the skin by aging were improved to make skin firm and beautiful, as well as its makeup effect.

The cosmetic for prevention of skin aging and the agent for prevention of skin aging in accordance with the present invention achieve an excellent effect in the improvement of wrinkles and sagging of the skin caused by intrinsic aging, by exposure to active oxygen species such as singlet oxygen or to ultraviolet ray, etc.

Accordingly, the cosmetic for prevention of skin aging, etc. in accordance with the present invention can be advantageously used as various cosmetics such as cosmetic with an object of prevention of aging including cosmetic of an emulsified type (such as milky lotion and cream) as well as lotion, facial pack, cleansing agent, makeup cosmetic, dispersed liquid, etc.

What is claimed is:

1. A cosmetic for prevention of skin aging which is characterized in containing the following components (A) and (B):
(A) L-histidine: and
(B) one or more active ingredient(s) selected from a group consisting of comfrey extract, yeast extract and ceramide.

2. The cosmetic for prevention of skin aging according to claim 1, wherein there are contained 0.001-5.0% by weight of the component (A) to the total amount of the cosmetic and 0.001-10% by weight of the component (B) comprising at least one of comfrey extract, yeast extract and ceramide to the total amount of the cosmetic.

3. The cosmetic for prevention of skin aging according to claim 1, wherein comfrey extract, yeast extract and ceramide are contained as the component (B).

4. The cosmetic for prevention of skin aging according to claim 1, wherein it is a cosmetic of an emulsified type.

5. An agent for prevention of skin aging which is characterized in containing the following components (A) and (B):
(A) L-histidine; and
(B) one or more active ingredient(s) selected from a group consisting of comfrey extract, yeast extract and ceramide.

6. A method for the prevention of skin aging, characterized in that, an agent for prevention of skin aging which is characterized in containing the following components (A) and (B) is applied to the skin.
(A) L-histidine; and
(B) one or more active ingredient(s) selected from a group consisting of comfrey extract, yeast extract and ceramide.
